(19) United States
(12) Patent Application Publication
(10) Pub. No.: US 2026/0141455 A1
(43) Pub. Date: May 21, 2026

(54) LOCATION BASED DATABASE SYSTEM DASHBOARDS

(71) Applicant: Express Scripts Strategic Development, Inc., St. Louis, MO (US)

(72) Inventors: Aanal Patel, Bridgewater, NJ (US); Pritesh J. Shah, Paramus, NJ (US); Ria Westergaard, Caledonia, IL (US); Harshavardhan Ramamurthy, Mandya (IN)

(21) Appl. No.: 18/954,620

(22) Filed: Nov. 21, 2024

(51) Int. Cl. G06Q 40/08; G16H 10/20; G16H 10/60

(52) U.S. Cl. CPC G06Q 40/08; G16H 10/20; G16H 10/60

(57) ABSTRACT

An example method for implementing location based dashboards for clinical trials includes obtaining a target health condition, accessing patient medical claims data and patient prescription claims data stored in one or more databases. For each candidate patient in the set of candidate patients, the method includes removing the candidate patient from the set in response to the candidate patient having demographic data which matches demographic exclusion criteria, the candidate patient having medical history data which matches medical exclusion criteria, or a distance between the candidate patient and a clinical trial study site is greater than a specified distance threshold. The method includes storing a set of eligible candidate patients after selective removal of candidate patients, transmitting a communication to at least one computing device, and displaying the set of eligible candidate patients and the at least one study site on a dashboard map of a user interface.

### FIELD

The present disclosure relates to location based database system dashboards.

### BACKGROUND

Pharmaceuticals companies, research organizations, etc. have difficulty determining where the best locations are to conduct clinical trials, such as areas for potential study sites where there are a lot of eligible patients and providers (which may have some association with a target health condition of a clinical trial study).

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

An example computer system includes memory hardware configured to store one or more databases, the one or more databases including patient medical claims data and patient prescription claims data, and processor hardware configured to execute instructions to obtain a target health condition, wherein the target health condition is a subject of a clinical trial, access the patient medical claims data and the patient prescription claims data stored in the one or more databases, identify a set of candidate patients having at least one medical claim associated with the target health condition, based on the patient medical claim data, update the set of candidate patients to include patients having at least one prescription claim associated with treatment of the target health condition, based on the patient prescription claim data, for each candidate patient in the set of candidate patients, remove the candidate patient from the set in response to the candidate patient having demographic data which matches demographic exclusion criteria, remove the candidate patient from the set in response to the candidate patient having medical history data which matches medical exclusion criteria, remove the candidate patient from the set in response to health insurance coverage data of the candidate patient failing to satisfy health insurance eligibility criteria, determine a distance between a location of the candidate patient and a location of at least one study site of the clinical trial, and remove the candidate patient from the set when the distance is greater than a specified distance threshold, store a set of eligible candidate patients after selective removal of candidate patients based on the demographic exclusion criteria, the medical exclusion criteria, the health insurance eligibility criteria and the specified distance threshold, transmit a communication to at least one computing device, the communication including the set of eligible candidate patients, and display the set of eligible candidate patients and the at least one study site on a dashboard map of a user interface.

In some examples, the processor hardware is configured to execute instructions to remove the candidate patient from the set in response to the candidate patient having medical history data which matches medical exclusion criteria by comparing prescription claims data of the candidate patient to a list of excluded prescription claims, comparing medical codes associated with the candidate patient to a list of excluded medical codes, and comparing medical claims data of the candidate patient to a list of excluded medical claims.

In some examples, the list of excluded prescription claims includes a prescription drug code for at least one of ocrelizumab, rituximab, belimumab, or anifrolumab.

In some examples, the list of excluded medical claims includes a medical claim code for at least one of human immunodeficiency virus disease, aquired absence of spleen, acute hepatitis B, acute hepatitis C, chronic hepatitis B, chronic hepatitis C, or history of malignancy except cervical cancer, basal cell carcinoma, squamous cell carcinoma or anifrolumab.

In some examples, identifying a set of candidate patients having at least one prescription claim associated with treatment of the target health condition includes a prescription drug code for at least one of oral corticosteroids, chloroquine, hydroxychloroquine, mycophenolate mofetil, mycophenolic acid, azathioprine, mercaptopurine, or methotrexate.

In some examples, the target health condition is Systemic Lupus Erythematosus. In some examples, determining the distance between the location of the candidate patient and the location of at least one study site includes using a cosine similarity function.

In some examples, the specified distance threshold is less than or equal to fifty miles. In some examples, the demographic exclusion criteria includes a patient age range of greater than or equal to eighteen years of age and less than or equal to seventy years of age.

In some examples, the location of the candidate patient and the location of at least one study site are masked zip code locations, and the masked zip code locations have the last two digits of each zip code masked.

In some examples, the processor hardware is configured to, for each candidate patient in the set of candidate patients, remove the candidate patient from the set in response to the candidate patient having a zip code matching a list of specified sensitive zip codes.

In some examples, the processor hardware is configured to execute instructions to obtain a set of providers associated with the set of eligible candidate patients, identify a set of candidate providers having at least one medical claim diagnosis associated with the target health condition, based on provider diagnosis data, and update the set of candidate providers to include providers having proscribed at least one prescription claim associated with treatment of the target health condition, based on provider prescription data.

In some examples, the processor hardware is configured to execute instructions to, for each candidate provider in the set of candidate providers, determine a distance between a location of the candidate provider and the location of the at least one study site of the clinical trial, and remove the candidate provider from the set when the distance is greater than the specified distance threshold.

An example method for implementing location based dashboards for clinical trials includes obtaining a target health condition, wherein the target health condition is a subject of a clinical trial, accessing patient medical claims data and patient prescription claims data stored in one or more databases, identifying a set of candidate patients having at least one medical claim associated with the target health condition, based on the patient medical claim data, updating the set of candidate patients to include patients having at least one prescription claim associated with treatment of the target health condition, based on the patient prescription claim data, for each candidate patient in the set of candidate patients, removing the candidate patient from the set in response to the candidate patient having demographic data which matches demographic exclusion criteria, removing the candidate patient from the set in response to the candidate patient having medical history data which matches medical exclusion criteria, removing the candidate patient from the set in response to health insurance coverage data of the candidate patient failing to satisfy health insurance eligibility criteria, determining a distance between a location of the candidate patient and a location of at least one study site of the clinical trial, and removing the candidate patient from the set when the distance is greater than a specified distance threshold, storing a set of eligible candidate patients after selective removal of candidate patients based on the demographic exclusion criteria, the medical exclusion criteria, the health insurance eligibility criteria and the specified distance threshold, transmitting a communication to at least one computing device, the communication including the set of eligible candidate patients, and displaying the set of eligible candidate patients and the at least one study site on a dashboard map of a user interface.

In some examples, removing the candidate patient from the set in response to the candidate patient having medical history data which matches medical exclusion criteria includes comparing prescription claims data of the candidate patient to a list of excluded prescription claims, comparing medical codes associated with the candidate patient to a list of excluded medical codes, and comparing medical claims data of the candidate patient to a list of excluded medical claims.

In some examples, the list of excluded prescription claims includes a prescription drug code for at least one of ocrelizumab, rituximab, belimumab, or anifrolumab.

In some examples, the list of excluded medical claims includes a medical claim code for at least one of human immunodeficiency virus disease, aquired absence of spleen, acute hepatitis B, acute hepatitis C, chronic hepatitis B, chronic hepatitis C, or history of malignancy except cervical cancer, basal cell carcinoma, squamous cell carcinoma or anifrolumab.

In some examples, identifying a set of candidate patients having at least one prescription claim associated with treatment of the target health condition includes a prescription drug code for at least one of oral corticosteroids, chloroquine, hydroxychloroquine, mycophenolate mofetil, mycophenolic acid, azathioprine, mercaptopurine, or methotrexate.

In some examples, the target health condition is Systemic Lupus Erythematosus. In some examples, determining the distance between the location of the candidate patient and the location of at least one study site includes using a cosine similarity function.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 is a functional block diagram of an example system including a high-volume pharmacy.

FIG. 2 is a functional block diagram of an example pharmacy fulfillment device, which may be deployed within the system of FIG. 1.

FIG. 3 is a functional block diagram of an example order processing device, which may be deployed within the system of FIG. 1.

FIG. 4 is a functional block diagram of an example database system including location based dashboards for clinical trials.

FIG. 5 is a message sequence chart illustrating interactions between components of the database system of FIG. 4.

FIG. 6 is a flowchart depicting an example process for identifying target patients using a location based dashboard.

FIG. 7 is a flowchart depicting an example process for determining distances between target patients and clinical trial study sites based on location data.

FIG. 8 is a flowchart depicting an example process for identifying target prescribers using a location based dashboard.

FIG. 9 is a flowchart depicting an example process for determining distances between target prescribers and clinical trial study sites based on location data.

In the drawings, reference numbers may be reused to identify similar and/or identical elements.

### DETAILED DESCRIPTION

### High-Volume Pharmacy

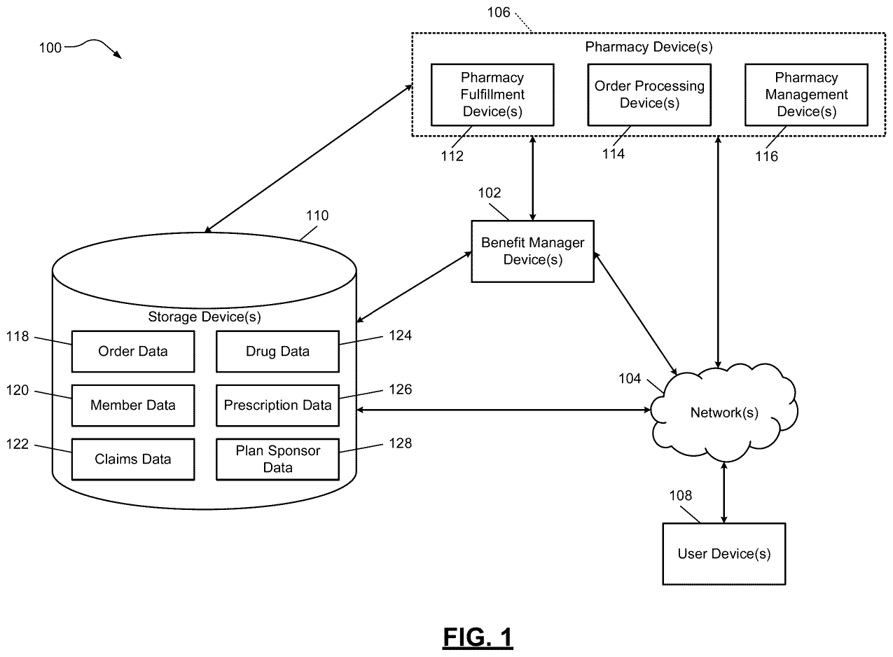

FIG. 1 is a block diagram of an example implementation of a system 100 for a high-volume pharmacy. While the system 100 is generally described as being deployed in a high-volume pharmacy or a fulfillment center (for example, a mail order pharmacy, a direct delivery pharmacy, etc.), the system 100 and/or components of the system 100 may otherwise be deployed (for example, in a lower-volume pharmacy, etc.). A high-volume pharmacy may be a pharmacy that is capable of filling at least some prescriptions mechanically. The system 100 may include a benefit manager device 102 and a pharmacy device 106 in communication with each other directly and/or over a network 104.

The system 100 may also include one or more user device(s) 108. A user, such as a pharmacist, patient, data analyst, health plan administrator, etc., may access the benefit manager device 102 or the pharmacy device 106 using the user device 108. The user device 108 may be a desktop computer, a laptop computer, a tablet, a smartphone, etc.

The benefit manager device 102 is a device operated by an entity that is at least partially responsible for creation and/or management of the pharmacy or drug benefit. While the entity operating the benefit manager device 102 is typically a pharmacy benefit manager (PBM), other entities may operate the benefit manager device 102 on behalf of themselves or other entities (such as PBMs). For example, the benefit manager device 102 may be operated by a health plan, a retail pharmacy chain, a drug wholesaler, a data analytics or other type of software-related company, etc. In some implementations, a PBM that provides the pharmacy benefit may provide one or more additional benefits including a medical or health benefit, a dental benefit, a vision benefit, a wellness benefit, a radiology benefit, a pet care benefit, an insurance benefit, a long term care benefit, a nursing home benefit, etc. The PBM may, in addition to its PBM operations, operate one or more pharmacies. The pharmacies may be retail pharmacies, mail order pharmacies, etc.

Some of the operations of the PBM that operates the benefit manager device 102 may include the following activities and processes. A member (or a person on behalf of the member) of a pharmacy benefit plan may obtain a prescription drug at a retail pharmacy location (e.g., a location of a physical store) from a pharmacist or a pharmacist technician. The member may also obtain the prescription drug through mail order drug delivery from a mail order pharmacy location, such as the system 100. In some implementations, the member may obtain the prescription drug directly or indirectly through the use of a machine, such as a kiosk, a vending unit, a mobile electronic device, or a different type of mechanical device, electrical device, electronic communication device, and/or computing device. Such a machine may be filled with the prescription drug in prescription packaging, which may include multiple prescription components, by the system 100. The pharmacy benefit plan is administered by or through the benefit manager device 102.

The member may have a copayment for the prescription drug that reflects an amount of money that the member is responsible to pay the pharmacy for the prescription drug. The money paid by the member to the pharmacy may come from, as examples, personal funds of the member, a health savings account (HSA) of the member or the member's family, a health reimbursement arrangement (HRA) of the member or the member's family, or a flexible spending account (FSA) of the member or the member's family. In some instances, an employer of the member may directly or indirectly fund or reimburse the member for the copayments.

The amount of the copayment required by the member may vary across different pharmacy benefit plans having different plan sponsors or clients and/or for different prescription drugs. The member's copayment may be a flat copayment (in one example, $10), coinsurance (in one example, 10%), and/or a deductible (for example, responsibility for the first $500 of annual prescription drug expense, etc.) for certain prescription drugs, certain types and/or classes of prescription drugs, and/or all prescription drugs. The copayment may be stored in a storage device 110 or determined by the benefit manager device 102.

In some instances, the member may not pay the copayment or may only pay a portion of the copayment for the prescription drug. For example, if a usual and customary cost for a generic version of a prescription drug is $4, and the member's flat copayment is $20 for the prescription drug, the member may only need to pay $4 to receive the prescription drug. In another example involving a worker's compensation claim, no copayment may be due by the member for the prescription drug.

In addition, copayments may also vary based on different delivery channels for the prescription drug. For example, the copayment for receiving the prescription drug from a mail order pharmacy location may be less than the copayment for receiving the prescription drug from a retail pharmacy location.

In conjunction with receiving a copayment (if any) from the member and dispensing the prescription drug to the member, the pharmacy submits a claim to the PBM for the prescription drug. After receiving the claim, the PBM (such as by using the benefit manager device 102) may perform certain adjudication operations including verifying eligibility for the member, identifying/reviewing an applicable formulary for the member to determine any appropriate copayment, coinsurance, and deductible for the prescription drug, and performing a drug utilization review (DUR) for the member. Further, the PBM may provide a response to the pharmacy (for example, the pharmacy system 100) following performance of at least some of the aforementioned operations.

As part of the adjudication, a plan sponsor (or the PBM on behalf of the plan sponsor) ultimately reimburses the pharmacy for filling the prescription drug when the prescription drug is successfully adjudicated. The aforementioned adjudication operations generally occur before the copayment is received and the prescription drug is dispensed. However, in some instances, these operations may occur simultaneously, substantially simultaneously, or in a different order. In addition, more or fewer adjudication operations may be performed as at least part of the adjudication process.

The amount of reimbursement paid to the pharmacy by a plan sponsor and/or money paid by the member may be determined at least partially based on types of pharmacy networks in which the pharmacy is included. In some implementations, the amount may also be determined based on other factors. For example, if the member pays the pharmacy for the prescription drug without using the prescription or drug benefit provided by the PBM, the amount of money paid by the member may be higher than when the member uses the prescription or drug benefit. In some implementations, the amount of money received by the pharmacy for dispensing the prescription drug and for the prescription drug itself may be higher than when the member uses the prescription or drug benefit. Some or all of the foregoing operations may be performed by executing instructions stored in the benefit manager device 102 and/or an additional device.

Examples of the network 104 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 802.11 standards network, as well as various combinations of the above networks. The network 104 may include an optical network. The network 104 may be a local area network or a global communication network, such as the Internet. In some implementations, the network 104 may include a network dedicated to prescription orders: a prescribing network such as the electronic prescribing network operated by Surescripts of Arlington, Virginia.

Moreover, although the system shows a single network 104, multiple networks can be used. The multiple networks may communicate in series and/or parallel with each other to link the devices 102-110.

The pharmacy device 106 may be a device associated with a retail pharmacy location (e.g., an exclusive pharmacy location, a grocery store with a retail pharmacy, or a general sales store with a retail pharmacy) or other type of pharmacy location at which a member attempts to obtain a prescription. The pharmacy may use the pharmacy device 106 to submit the claim to the PBM for adjudication.

Additionally, in some implementations, the pharmacy device 106 may enable information exchange between the pharmacy and the PBM. For example, this may allow the sharing of member information such as drug history that may allow the pharmacy to better service a member (for example, by providing more informed therapy consultation and drug interaction information). In some implementations, the benefit manager device 102 may track prescription drug fulfillment and/or other information for users that are not members, or have not identified themselves as members, at the time (or in conjunction with the time) in which they seek to have a prescription filled at a pharmacy.

The pharmacy device 106 may include a pharmacy fulfillment device 112, an order processing device 114, and a pharmacy management device 116 in communication with each other directly and/or over the network 104. The order processing device 114 may receive information regarding filling prescriptions and may direct an order component to one or more devices of the pharmacy fulfillment device 112 at a pharmacy. The pharmacy fulfillment device 112 may fulfill, dispense, aggregate, and/or pack the order components of the prescription drugs in accordance with one or more prescription orders directed by the order processing device 114.

In general, the order processing device 114 is a device located within or otherwise associated with the pharmacy to enable the pharmacy fulfillment device 112 to fulfill a prescription and dispense prescription drugs. In some implementations, the order processing device 114 may be an external order processing device separate from the pharmacy and in communication with other devices located within the pharmacy.

For example, the external order processing device may communicate with an internal pharmacy order processing device and/or other devices located within the system 100. In some implementations, the external order processing device may have limited functionality (e.g., as operated by a user requesting fulfillment of a prescription drug), while the internal pharmacy order processing device may have greater functionality (e.g., as operated by a pharmacist).

The order processing device 114 may track the prescription order as it is fulfilled by the pharmacy fulfillment device 112. The prescription order may include one or more prescription drugs to be filled by the pharmacy. The order processing device 114 may make pharmacy routing decisions and/or order consolidation decisions for the particular prescription order. The pharmacy routing decisions include what device(s) in the pharmacy are responsible for filling or otherwise handling certain portions of the prescription order. The order consolidation decisions include whether portions of one prescription order or multiple prescription orders should be shipped together for a user or a user family. The order processing device 114 may also track and/or schedule literature or paperwork associated with each prescription order or multiple prescription orders that are being shipped together. In some implementations, the order processing device 114 may operate in combination with the pharmacy management device 116.

The order processing device 114 may include circuitry, a processor, a memory to store data and instructions, and communication functionality. The order processing device 114 is dedicated to performing processes, methods, and/or instructions described in this application. Other types of electronic devices may also be used that are specifically configured to implement the processes, methods, and/or instructions described in further detail below.

In some implementations, at least some functionality of the order processing device 114 may be included in the pharmacy management device 116. The order processing device 114 may be in a client-server relationship with the pharmacy management device 116, in a peer-to-peer relationship with the pharmacy management device 116, or in a different type of relationship with the pharmacy management device 116. The order processing device 114 and/or the pharmacy management device 116 may communicate directly (for example, such as by using a local storage) and/or through the network 104 (such as by using a cloud storage configuration, software as a service, etc.) with the storage device 110.

The storage device 110 may include: non-transitory storage (for example, memory, hard disk, CD-ROM, etc.) in communication with the benefit manager device 102 and/or the pharmacy device 106 directly and/or over the network 104. The non-transitory storage may store order data 118, member data 120, claims data 122, drug data 124, prescription data 126, and/or plan sponsor data 128. Further, the system 100 may include additional devices, which may communicate with each other directly or over the network 104.

The order data 118 may be related to a prescription order. The order data may include type of the prescription drug (for example, drug name and strength) and quantity of the prescription drug. The order data 118 may also include data used for completion of the prescription, such as prescription materials. In general, prescription materials include an electronic copy of information regarding the prescription drug for inclusion with or otherwise in conjunction with the fulfilled prescription. The prescription materials may include electronic information regarding drug interaction warnings, recommended usage, possible side effects, expiration date, date of prescribing, etc. The order data 118 may be used by a high-volume fulfillment center to fulfill a pharmacy order.

In some implementations, the order data 118 includes verification information associated with fulfillment of the prescription in the pharmacy. For example, the order data 118 may include videos and/or images taken of (i) the prescription drug prior to dispensing, during dispensing, and/or after dispensing, (ii) the prescription container (for example, a prescription container and sealing lid, prescription packaging, etc.) used to contain the prescription drug prior to dispensing, during dispensing, and/or after dispensing, (iii) the packaging and/or packaging materials used to ship or otherwise deliver the prescription drug prior to dispensing, during dispensing, and/or after dispensing, and/or (iv) the fulfillment process within the pharmacy. Other types of verification information such as barcode data read from pallets, bins, trays, or carts used to transport prescriptions within the pharmacy may also be stored as order data 118.

The member data 120 includes information regarding the members associated with the PBM. The information stored as member data 120 may include personal information, personal health information, protected health information, etc. Examples of the member data 120 include name, address, telephone number, e-mail address, prescription drug history, etc. The member data 120 may include a plan sponsor identifier that identifies the plan sponsor associated with the member and/or a member identifier that identifies the member to the plan sponsor. The member data 120 may include a member identifier that identifies the plan sponsor associated with the user and/or a user identifier that identifies the user to the plan sponsor. The member data 120 may also include dispensation preferences such as type of label, type of cap, message preferences, language preferences, etc.

The member data 120 may be accessed by various devices in the pharmacy (for example, the high-volume fulfillment center, etc.) to obtain information used for fulfillment and shipping of prescription orders. In some implementations, an external order processing device operated by or on behalf of a member may have access to at least a portion of the member data 120 for review, verification, or other purposes.

In some implementations, the member data 120 may include information for persons who are users of the pharmacy but are not members in the pharmacy benefit plan being provided by the PBM. For example, these users may obtain drugs directly from the pharmacy, through a private label service offered by the pharmacy, the high-volume fulfillment center, or otherwise. In general, the terms “member” and “user” may be used interchangeably.

The claims data 122 includes information regarding pharmacy claims adjudicated by the PBM under a drug benefit program provided by the PBM for one or more plan sponsors. In general, the claims data 122 includes an identification of the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copayment/coinsurance amount, rebate information, and/or member eligibility, etc. Additional information may be included.

In some implementations, other types of claims beyond prescription drug claims may be stored in the claims data 122. For example, medical claims, dental claims, wellness claims, or other types of health-care-related claims for members may be stored as a portion of the claims data 122.

In some implementations, the claims data 122 includes claims that identify the members with whom the claims are associated. Additionally, or alternatively, the claims data 122 may include claims that have been de-identified (that is, associated with a unique identifier but not with a particular, identifiable member).

The drug data 124 may include drug name (e.g., technical name and/or common name), other names by which the drug is known, active ingredients, an image of the drug (such as in pill form), etc. The drug data 124 may include information associated with a single medication or multiple medications.

The prescription data 126 may include information regarding prescriptions that may be issued by prescribers on behalf of users, who may be members of the pharmacy benefit plan—for example, to be filled by a pharmacy. Examples of the prescription data 126 include user names, medication or treatment (such as lab tests), dosing information, etc. The prescriptions may include electronic prescriptions or paper prescriptions that have been scanned. In some implementations, the dosing information reflects a frequency of use (e.g., once a day, twice a day, before each meal, etc.) and a duration of use (e.g., a few days, a week, a few weeks, a month, etc.).

In some implementations, the order data 118 may be linked to associated member data 120, claims data 122, drug data 124, and/or prescription data 126.

The plan sponsor data 128 includes information regarding the plan sponsors of the PBM. Examples of the plan sponsor data 128 include company name, company address, contact name, contact telephone number, contact e-mail address, etc.

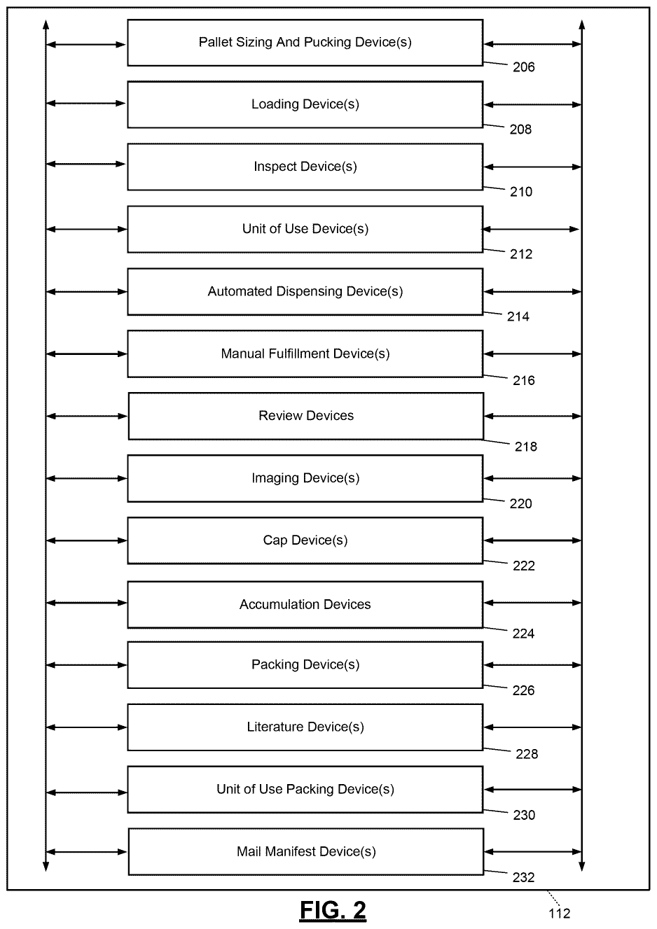

FIG. 2 illustrates the pharmacy fulfillment device 112 according to an example implementation. The pharmacy fulfillment device 112 may be used to process and fulfill prescriptions and prescription orders. After fulfillment, the fulfilled prescriptions are packed for shipping.

The pharmacy fulfillment device 112 may include devices in communication with the benefit manager device 102, the order processing device 114, and/or the storage device 110, directly or over the network 104. Specifically, the pharmacy fulfillment device 112 may include pallet sizing and pucking device(s) 206, loading device(s) 208, inspect device(s) 210, unit of use device(s) 212, automated dispensing device(s) 214, manual fulfillment device(s) 216, review devices 218, imaging device(s) 220, cap device(s) 222, accumulation devices 224, packing device(s) 226, literature device(s) 228, unit of use packing device(s) 230, and mail manifest device(s) 232. Further, the pharmacy fulfillment device 112 may include additional devices, which may communicate with each other directly or over the network 104.

In some implementations, operations performed by one of these devices 206-232 may be performed sequentially, or in parallel with the operations of another device as may be coordinated by the order processing device 114. In some implementations, the order processing device 114 tracks a prescription with the pharmacy based on operations performed by one or more of the devices 206-232.

In some implementations, the pharmacy fulfillment device 112 may transport prescription drug containers, for example, among the devices 206-232 in the high-volume fulfillment center, by use of pallets. The pallet sizing and pucking device 206 may configure pucks in a pallet. A pallet may be a transport structure for a number of prescription containers, and may include a number of cavities. A puck may be placed in one or more than one of the cavities in a pallet by the pallet sizing and pucking device 206. The puck may include a receptacle sized and shaped to receive a prescription container. Such containers may be supported by the pucks during carriage in the pallet. Different pucks may have differently sized and shaped receptacles to accommodate containers of differing sizes, as may be appropriate for different prescriptions.

The arrangement of pucks in a pallet may be determined by the order processing device 114 based on prescriptions that the order processing device 114 decides to launch. The arrangement logic may be implemented directly in the pallet sizing and pucking device 206. Once a prescription is set to be launched, a puck suitable for the appropriate size of container for that prescription may be positioned in a pallet by a robotic arm or pickers. The pallet sizing and pucking device 206 may launch a pallet once pucks have been configured in the pallet.

The loading device 208 may load prescription containers into the pucks on a pallet by a robotic arm, a pick and place mechanism (also referred to as pickers), etc. In various implementations, the loading device 208 has robotic arms or pickers to grasp a prescription container and move it to and from a pallet or a puck. The loading device 208 may also print a label that is appropriate for a container that is to be loaded onto the pallet, and apply the label to the container. The pallet may be located on a conveyor assembly during these operations (e.g., at the high-volume fulfillment center, etc.).

The inspect device 210 may verify that containers in a pallet are correctly labeled and in the correct spot on the pallet. The inspect device 210 may scan the label on one or more containers on the pallet. Labels of containers may be scanned or imaged in full or in part by the inspect device 210. Such imaging may occur after the container has been lifted out of its puck by a robotic arm, picker, etc., or may be otherwise scanned or imaged while retained in the puck. In some implementations, images and/or video captured by the inspect device 210 may be stored in the storage device 110 as order data 118.

The unit of use device 212 may temporarily store, monitor, label, and/or dispense unit of use products. In general, unit of use products are prescription drug products that may be delivered to a user or member without being repackaged at the pharmacy. These products may include pills in a container, pills in a blister pack, inhalers, etc. Prescription drug products dispensed by the unit of use device 212 may be packaged individually or collectively for shipping, or may be shipped in combination with other prescription drugs dispensed by other devices in the high-volume fulfillment center.

At least some of the operations of the devices 206-232 may be directed by the order processing device 114. For example, the manual fulfillment device 216, the review device 218, the automated dispensing device 214, and/or the packing device 226, etc. may receive instructions provided by the order processing device 114.

The automated dispensing device 214 may include one or more devices that dispense prescription drugs or pharmaceuticals into prescription containers in accordance with one or multiple prescription orders. In general, the automated dispensing device 214 may include mechanical and electronic components with, in some implementations, software and/or logic to facilitate pharmaceutical dispensing that would otherwise be performed in a manual fashion by a pharmacist and/or pharmacist technician. For example, the automated dispensing device 214 may include high-volume fillers that fill a number of prescription drug types at a rapid rate and blister pack machines that dispense and pack drugs into a blister pack. Prescription drugs dispensed by the automated dispensing devices 214 may be packaged individually or collectively for shipping, or may be shipped in combination with other prescription drugs dispensed by other devices in the high-volume fulfillment center.

The manual fulfillment device 216 controls how prescriptions are manually fulfilled. For example, the manual fulfillment device 216 may receive or obtain a container and enable fulfillment of the container by a pharmacist or pharmacy technician. In some implementations, the manual fulfillment device 216 provides the filled container to another device in the pharmacy fulfillment devices 112 to be joined with other containers in a prescription order for a user or member.

In general, manual fulfillment may include operations at least partially performed by a pharmacist or a pharmacy technician. For example, a person may retrieve a supply of the prescribed drug, may make an observation, may count out a prescribed quantity of drugs and place them into a prescription container, etc. Some portions of the manual fulfillment process may be automated by use of a machine. For example, counting of capsules, tablets, or pills may be at least partially automated (such as through use of a pill counter). Prescription drugs dispensed by the manual fulfillment device 216 may be packaged individually or collectively for shipping, or may be shipped in combination with other prescription drugs dispensed by other devices in the high-volume fulfillment center.

The review device 218 may process prescription containers to be reviewed by a pharmacist for proper pill count, exception handling, prescription verification, etc. Fulfilled prescriptions may be manually reviewed and/or verified by a pharmacist, as may be required by state or local law. A pharmacist or other licensed pharmacy person who may dispense certain drugs in compliance with local and/or other laws may operate the review device 218 and visually inspect a prescription container that has been filled with a prescription drug. The pharmacist may review, verify, and/or evaluate drug quantity, drug strength, and/or drug interaction concerns, or otherwise perform pharmacist services. The pharmacist may also handle containers which have been flagged as an exception, such as containers with unreadable labels, containers for which the associated prescription order has been canceled, containers with defects, etc. In an example, the manual review can be performed at a manual review station.

The imaging device 220 may image containers once they have been filled with pharmaceuticals. The imaging device 220 may measure a fill height of the pharmaceuticals in the container based on the obtained image to determine if the container is filled to the correct height given the type of pharmaceutical and the number of pills in the prescription. Images of the pills in the container may also be obtained to detect the size of the pills themselves and markings thereon. The images may be transmitted to the order processing device 114 and/or stored in the storage device 110 as part of the order data 118.

The cap device 222 may be used to cap or otherwise seal a prescription container. In some implementations, the cap device 222 may secure a prescription container with a type of cap in accordance with a user preference (e.g., a preference regarding child resistance, etc.), a plan sponsor preference, a prescriber preference, etc. The cap device 222 may also etch a message into the cap, although this process may be performed by a subsequent device in the high-volume fulfillment center.

The accumulation device 224 accumulates various containers of prescription drugs in a prescription order. The accumulation device 224 may accumulate prescription containers from various devices or areas of the pharmacy. For example, the accumulation device 224 may accumulate prescription containers from the unit of use device 212, the automated dispensing device 214, the manual fulfillment device 216, and the review device 218. The accumulation device 224 may be used to group the prescription containers prior to shipment to the member.

The literature device 228 prints, or otherwise generates, literature to include with each prescription drug order. The literature may be printed on multiple sheets of substrates, such as paper, coated paper, printable polymers, or combinations of the above substrates. The literature printed by the literature device 228 may include information required to accompany the prescription drugs included in a prescription order, other information related to prescription drugs in the order, financial information associated with the order (for example, an invoice or an account statement), etc.

In some implementations, the literature device 228 folds or otherwise prepares the literature for inclusion with a prescription drug order (e.g., in a shipping container). In other implementations, the literature device 228 prints the literature and is separate from another device that prepares the printed literature for inclusion with a prescription order.

The packing device 226 packages the prescription order in preparation for shipping the order. The packing device 226 may box, bag, or otherwise package the fulfilled prescription order for delivery. The packing device 226 may further place inserts (e.g., literature or other papers, etc.) into the packaging received from the literature device 228. For example, bulk prescription orders may be shipped in a box, while other prescription orders may be shipped in a bag, which may be a wrap seal bag.

The packing device 226 may label the box or bag with an address and a recipient's name. The label may be printed and affixed to the bag or box, be printed directly onto the bag or box, or otherwise associated with the bag or box. The packing device 226 may sort the box or bag for mailing in an efficient manner (e.g., sort by delivery address, etc.). The packing device 226 may include ice or temperature sensitive elements for prescriptions that are to be kept within a temperature range during shipping (for example, this may be necessary in order to retain efficacy). The ultimate package may then be shipped through postal mail, through a mail order delivery service that ships via ground and/or air (e.g., UPS, FEDEX, or DHL, etc.), through a delivery service, through a locker box at a shipping site (e.g., AMAZON locker or a PO Box, etc.), or otherwise.

The unit of use packing device 230 packages a unit of use prescription order in preparation for shipping the order. The unit of use packing device 230 may include manual scanning of containers to be bagged for shipping to verify each container in the order. In an example implementation, the manual scanning may be performed at a manual scanning station. The pharmacy fulfillment device 112 may also include a mail manifest device 232 to print mailing labels used by the packing device 226 and may print shipping manifests and packing lists.

While the pharmacy fulfillment device 112 in FIG. 2 is shown to include single devices 206-232, multiple devices may be used. When multiple devices are present, the multiple devices may be of the same device type or models, or may be a different device type or model. The types of devices 206-232 shown in FIG. 2 are example devices. In other configurations of the system 100, lesser, additional, or different types of devices may be included.

Moreover, multiple devices may share processing and/or memory resources. The devices 206-232 may be located in the same area or in different locations. For example, the devices 206-232 may be located in a building or set of adjoining buildings. The devices 206-232 may be interconnected (such as by conveyors), networked, and/or otherwise in contact with one another or integrated with one another (e.g., at the high-volume fulfillment center, etc.). In addition, the functionality of a device may be split among a number of discrete devices and/or combined with other devices.

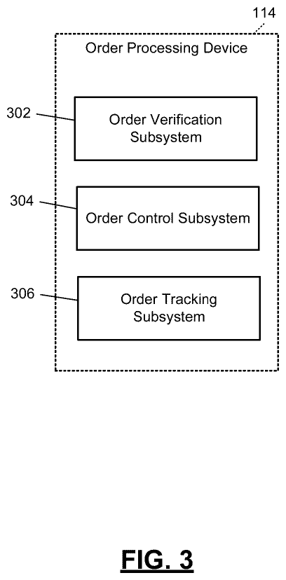

FIG. 3 illustrates the order processing device 114 according to an example implementation. The order processing device 114 may be used by one or more operators to generate prescription orders, make routing decisions, make prescription order consolidation decisions, track literature with the system 100, and/or view order status and other order related information. For example, the prescription order may be comprised of order components.

The order processing device 114 may receive instructions to fulfill an order without operator intervention. An order component may include a prescription drug fulfilled by use of a container through the system 100. The order processing device 114 may include an order verification subsystem 302, an order control subsystem 304, and/or an order tracking subsystem 306. Other subsystems may also be included in the order processing device 114.

The order verification subsystem 302 may communicate with the benefit manager device 102 to verify the eligibility of the member and review the formulary to determine appropriate copayment, coinsurance, and deductible for the prescription drug and/or perform a DUR (drug utilization review). Other communications between the order verification subsystem 302 and the benefit manager device 102 may be performed for a variety of purposes.

The order control subsystem 304 controls various movements of the containers and/or pallets along with various filling functions during their progression through the system 100. In some implementations, the order control subsystem 304 may identify the prescribed drug in one or more than one prescription orders as capable of being fulfilled by the automated dispensing device 214. The order control subsystem 304 may determine which prescriptions are to be launched and may determine that a pallet of automated-fill containers is to be launched.

The order control subsystem 304 may determine that an automated-fill prescription of a specific pharmaceutical is to be launched and may examine a queue of orders awaiting fulfillment for other prescription orders, which will be filled with the same pharmaceutical. The order control subsystem 304 may then launch orders with similar automated-fill pharmaceutical needs together in a pallet to the automated dispensing device 214. As the devices 206-232 may be interconnected by a system of conveyors or other container movement systems, the order control subsystem 304 may control various conveyors: for example, to deliver the pallet from the loading device 208 to the manual fulfillment device 216 from the literature device 228, paperwork as needed to fill the prescription.

The order tracking subsystem 306 may track a prescription order during its progress toward fulfillment. The order tracking subsystem 306 may track, record, and/or update order history, order status, etc. The order tracking subsystem 306 may store data locally (for example, in a memory) or as a portion of the order data 118 stored in the storage device 110.

### Location Based Database System Dashboards

Some example embodiments include user interfaces which display clinical trial locations across various geographic regions (e.g., on a dashboard), such as United States regions, states, counties, cities, zip codes, etc. Pharmaceuticals companies, research organizations, etc. have difficulty determining where the best locations are to conduct clinical trials, such as areas for potential study sites where there are a lot of eligible patients and providers (which may have some association with a target health condition of a clinical trial study).

In various implementations, a user interface may facilitate scaling down patient candidate data to different census tracts, and may use one or more socioeconomic indicators for each census tract, such as the Evernorth social determinants index (ESDI). Each potential patient within a census tract may be assigned the ESDI for the census tract in which they reside.

As an example, if a client sponsor would like to find patient candidates for a Lupus study, a user interface may display locations on a map (e.g., red squares on a dashboard) where study sites already exist. If there is a threshold number of existing patients diagnosed with Lupus or receiving treatment for Lupus, who are outside a specified distance of an existing study site (e.g., more than ten miles from an existing study site, more than fifty miles, etc.), the system may search for or display candidate providers to develop a new study site closer to those patients. In some example embodiments, the system may prioritize providers based on any suitable criteria, such as provider specialties, a number of patients within a specified area of the provider, etc.

A standard user interface may be presented to administrators of different clinical trials, while backend data may be customized based on a desired study (e.g., different target health conditions for various clinical trials). Candidate patients may be screened based on any suitable criteria, and Medical claims data and pharmacy claims data may be used to identify candidate patients for a study, and the candidate patients may be screened based on any suitable criteria (such as age or other demographic information, health insurance coverage eligibility, target health conditions to exclude from the study, etc.).

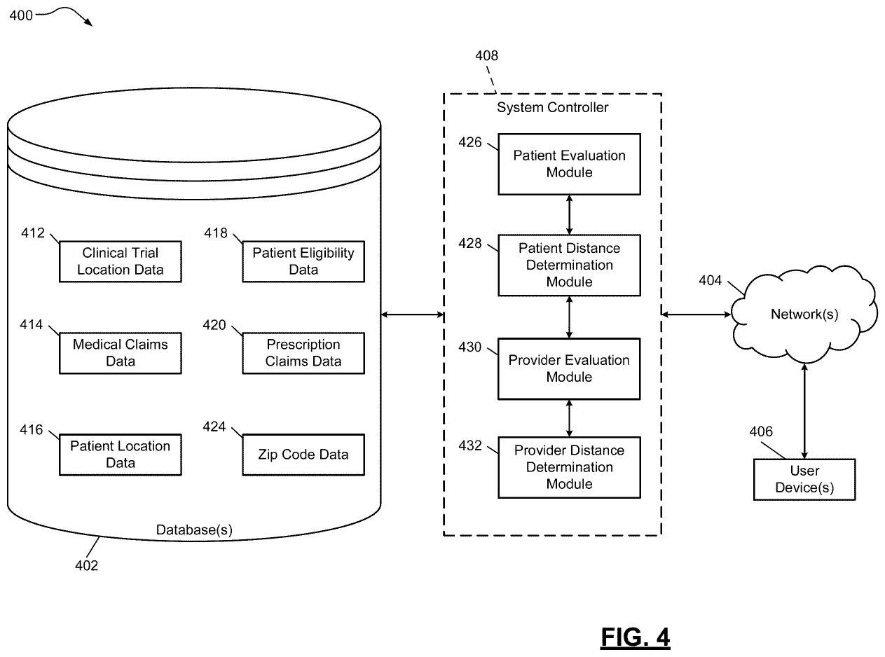

FIG. 4 is a functional block diagram of an example database system 400 including location based dashboards for clinical trials, which includes one or more databases 402. While the system 400 is generally described as being deployed in a computer network system, the database 402 and/or components of the system 400 may otherwise be deployed (for example, as a standalone computer setup). The system 400 may include a desktop computer, a laptop computer, a tablet, a smartphone, etc.

As shown in FIG. 4, the database 402 stores clinical trial location data 412, medical claims data 414, patient location data 416, patient eligibility data 418, prescription claims data 420, and zip code data 424. In various implementations, the database 402 may store other types of data as well, or may not store all of the example data types illustrated in FIG. 4.

The clinical trial location data 412, medical claims data 414, patient location data 416, patient eligibility data 418, prescription claims data 420, and zip code data 424 may be located in different physical memories within the database 402, such as different random access memory (RAM), read-only memory (ROM), a non-volatile hard disk or flash memory, etc., or be spread across multiple different databases. In some implementations, the clinical trial location data 412, medical claims data 414, patient location data 416, patient eligibility data 418, prescription claims data 420, and zip code data 424 may be located in the same memory (such as in different address ranges of the same memory). In various implementations, the clinical trial location data 412, medical claims data 414, patient location data 416, patient eligibility data 418, prescription claims data 420, and zip code data 424 may each be stored as structured or unstructured data in any suitable type of data store.

As shown in FIG. 4, a system controller 408 may include one or more modules, including a patient evaluation module 426, a patient distance determination module 428, a provider evaluation module 430, and a provider distance determination module 432 (e.g., where a provider may include a prescriber associated with patients). These example modules are provided for purposes of illustration, and other embodiments may include more or less modules, functions of different software features implemented in other modules or controller locations, more than one system controller, more than one database, data distributed to other databases, etc.

In various implementations, a system developer may access the system controller 408 via the user device 406. The user device 406 may include any suitable user device for displaying text and receiving input from a user, including a desktop computer, a laptop computer, a tablet, a smartphone, etc. In various implementations, the user device 406 may access the database 402 or the system controller 408 directly, or may access the database 402 or the system controller 408 through one or more networks 404. Example networks may include a wireless network, a local area network (LAN), the Internet, a cellular network, etc.

Clinical trial location data 412 may include any suitable data indicative of locations of clinical trial study sites, such as longitude and latitude data, GPS coordinates, zip code data, etc. The medical claims data 414 may include any suitable data regarding medical claims for patients, procedures performed for patients, medical diagnoses, etc.

The prescription claims data 420 may include any suitable prescription claims associated with patients, which may include prescriptions for specific drugs prescribed to treat health conditions or diseases, etc. The medical claims data 414 and the prescription claims data 420 may be associated with a target health condition of the clinical study or clinical trial.

Patient eligibility data 418 may include information about current health insurance coverage for patients, types of benefits provided under health insurance for patients, etc. The patient location data 416 may include any suitable data identifying a location of a patient or a patient's primary residence, such as latitude and longitude data, global positioning system (GPS) data, zip code data, census tract data, etc.

The zip code data 424 may include any suitable zip code data associated with patients, prescribers, providers, clinical trial study sites, etc. Zip code data may be encrypted or otherwise protected to protect sensitive patient data. For example, zip code data may have one or more digits masked (such as the last one, two or three digits), to provide privacy and security for sensitive patient data.

The patient evaluation module 426 may be configured to look for patients diagnosed with a target disease in last two years (or longer or shorter time periods), and also patients which have taken medications associated with treatment of the target disease. The patient evaluation module 426 may be configured to filter candidate patients based on age or other demographics, insurance plan eligibility, etc. Then patient evaluation module 426 may be configured to exclude some patients based on other drugs or medical codes associated with health conditions that the clinical trial administrator does not want to be part of the study, a distance from the study sites such as within fifty miles (or shorter or longer distances), etc.

The patient distance determination module 428 may be configured to determine distances from study sites for all candidate patients determined as eligible for the study after drug claim and medical claim filtering. For example, the patient distance determination module 428 may be configured to determine which filtered candidate patients are within fifty miles of a study site (or a shorter or longer distance).

Each study site may be uniquely identified by zip code or other location information. In some examples, sensitive zip codes may be filtered to protect patient privacy, such as zip codes having a population or area which is so small that patients could possibly be identified just by zip code information. Zip code data may be basked in some cases, such as using only the first three digits of zip code data to avoid identifying patient data.

In various implementations, a many-to-many combination of candidate patient locations may be compared to study site locations. For example, the system may check whether there are any matches between or more patients and one or more study sites within fifty miles (or a smaller or larger distance). If there are multiple matches, the system may select a closest study site to associate with each patient.

The system may be configured to generate any suitable output, such as a data table of entries including a patient identifier (ID), a study site ID, and an approximated distance between the patient and the study site. For example, there may be one row per patient in the table. This patient data may be used to obtain additional demographics and location information, and then the tables may be used to update a visualization map (e.g., showing a number or density of candidate patients within distance ranges of various study site locations).

The provider evaluation module 430 may be configured to operate similar to the patient evaluation module 426, but provider evaluation module 430 may query the claims differently (e.g., query the medical claims data and the prescription claims data to identify candidate patients associated with or diagnosed with a target health condition of the clinical trial).

For example, the provider evaluation module 430 may pull data based on providers, or only on carriers were the outreach is allowed. The provider evaluation module 430 may have a different view of similar medical claims and prescription claims data tables. The patient evaluation module 426 may only check data where the system is allowed to directly reach out to patients. The provider evaluation module 430 may have a larger number of candidate patient matches if it is not restricted to only patients that can be contacted directly. In this example, the target audience may be providers, where the system is merely evaluating candidate patients in the area of the providers.

In some examples, a clinical trial sponsor may want to know if there are providers near a study site, even if less patients are located near the site for direct contact outreach. The provider distance determination module 432 may be configured similar to the patient distance determination module 428, but based on locations of providers with respect to study sites. In some examples, the user interface (e.g., dashboard map) may illustrate patients both inside and outside a target distance range (such as fifty miles, or closer or further), using a different color or shape to indicate patients inside and outside of the target distance range.

In various implementations, any suitable database tools may be used by the system. For example, Teradata Sequel may be used for pulling data, as via one or more application programming interface (APIs) or representational state transfer (REST) APIs. A statistical package such as R may be used to determine distances between patients, study sites, and providers (e.g., prescribers).

Database processing may be optimized to facilitate space for pulling data and storing distance calculations, such as pulled data for fifteen to twenty thousand patients generating a distance calculation table of up to three-hundred thousand or more entries. In some examples, filtering candidate patients based on drug claim exclusions and other criteria, prior to performing distance calculations, may reduce the amount of computations and memory storage needed in the database, which increases speed of operation of the processer and system and improves memory storage space in the database system. For example, a distance calculation algorithm such as cosine similarity may be extremely computationally heavy, and example processes described herein which may keep distance estimation as a last step after filtering, may reduce processing demands on the system and speed up operation of the technical performance of the database system, including reduced memory usage.

Also, use of masked zip code data for distance calculations may provide a practical application of de-identifying location data to maintain patient confidentiality, while still allowing the system to perform distance calculations in view of the maintained patient confidentiality.

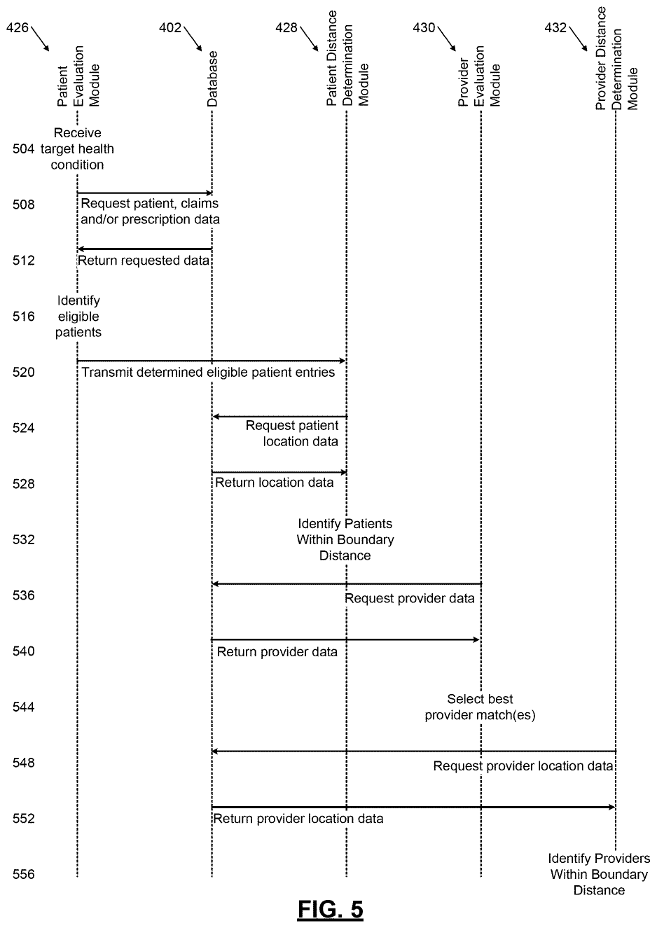

FIG. 5 is a message sequence chart illustrating interactions between components of the database system 400 of FIG. 4. For example, FIG. 5 illustrates example communications between the database 402, the patient evaluation module 426, the patient distance determination module 428, the provider evaluation module 430, and the provider distance determination module 432.

At line 504, the patient evaluation module is configured to receive a target health condition. For example, the target health condition may be a disease which is subject to a clinical trial at one or more study sites. At line 508, the patient evaluation module 426 requests, from the database 402, patient data, claims data, prescription data, etc.

At line 512, the database 402 returns the requested data to the patient evaluation module 426. At line 516, the patient evaluation module identifies eligible patients (e.g., candidates for the clinical trial), based on the requested data. For example, the determination of eligible patients may be based on patients having medical claims data and/or prescription drug claims data which corresponds to the target health condition of the clinical trial, and has occurred within a past specified period of time, such as the last two years, last one year, last six months, etc.

The Determination of eligible patients may also include other factors such as, whether the patient has experienced any medical conditions or prescription drug conditions which are excluded from the study, whether demographics of the patient are within a target range (such as an age range of above 18 years old and less than 70 years old), weather a patient's health insurance covers treatment for the target health condition of the clinical trial, etc.

At line 520, the patient evaluation module 426 transmits determined eligible patient entries (e.g., clinical trial candidate patients) to the patient distance determination module 428. At line 524, the patient distance determination module 428 requests patient location data from the database 402. The database 402 returns the requested patient location data to the patient distance determination module at line 528.

At line 532, the patient distance determination module 428 identifies patients within a specified boundary distance, such as within the nearest 10 miles, within the nearest 25 miles, within the nearest 50 miles, within the nearest 100 miles, etc. The determination of distance may be performed using any suitable algorithm, such as a cosine similarity function. The identification of the location of the patient may be based on, for example, stored latitude and longitude data of the patient, GPS coordinates for the patient and the patient's residence, zip code data of the patient (which may have one or more digits masked), etc.

At line 536, the provider evaluation module 430 requests provider data from the database 402. The database 402 then returns provider data to the provider evaluation module 430 at line 540. The provider evaluation module 430 determines the best provider matches at line 544. For example, the provider evaluation module 430 may identify prescribers who have diagnosed the target health condition of the clinical trial and/or prescription drugs associated with treatment of the target health condition, where the prescribers are optionally also associated with patients experiencing the target health condition and located within a specified proximity distance.

At line 548, the provider distance determination module 432 requests provider location data from the database 402. And at line 552, the database 402 returns provider location data to the provider distance determination module 432. For example, the provider location data may include GPS coordinates of a provider's office, latitude and longitude coordinates of the provider, GPS coordinates for the provider, etc. At line 556, the provider distance determination module 432 identifies providers within a boundary distance. For example, the provider distance determination module 432 may use a cosine similarity function (or other suitable algorithm) to determine which providers are within a specified distance of one or more study sites for a clinical trial, such as within fifty miles of distance between provider and the study site, etc.

### Processes for Location Based Database System Dashboards

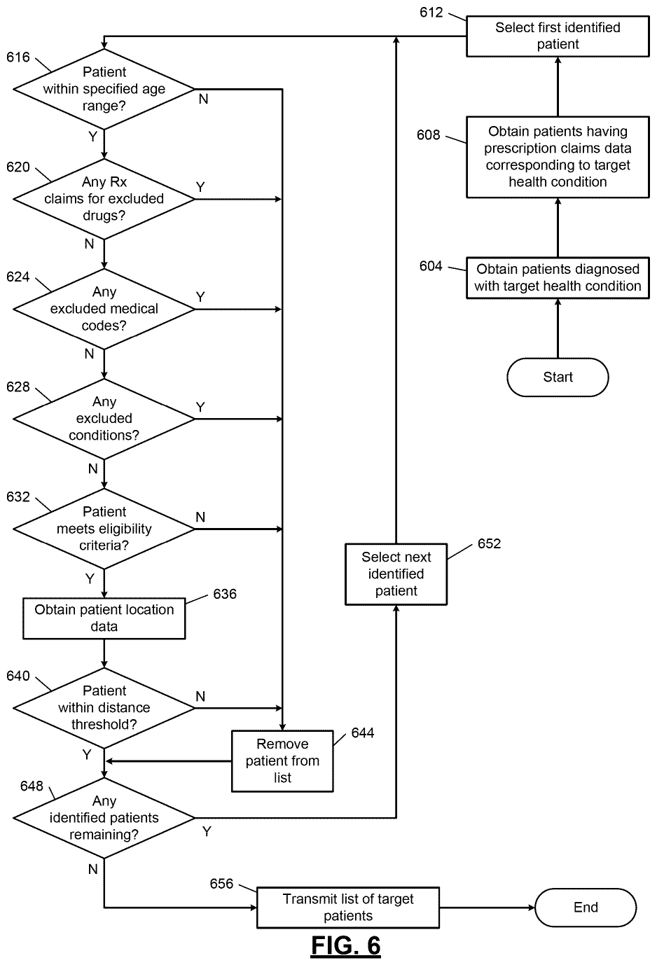

FIG. 6 is a flowchart depicting an example process for identifying candidate patients using a location based dashboard. In various implementations, the process of FIG. 5 may be executed by the system controller 408, such as by the patient evaluation module 426.

The process begins at 604, by obtaining patients diagnosed with a target health condition (e.g., a target health condition for study via a current or planned clinical trial). For example, the system controller 408 may be configured to identify patients who have medical claims data corresponding to the target health condition within a most recent specified time period, such as the last six months, last year, last two years, etc.

At 608, control identifies patients having prescription claims data corresponding to the target health condition. For example, the system controller may be configured to identify prescriptions which are related to treatment of the target health condition of the clinical trial, and identify patients having associated prescription claims. An example of prescription claim codes which may be used to identify patients for a target clinical study for, e.g., Systemic Lupus Erythematosus, is provided below is Table 1. The prescription drug claims in Table 1 are provided for example only, and other example embodiments may use other prescription drug claims associated with other target health conditions.

TABLE 1

| Medication | Parameter |
|---|---|
| oral corticosteroids | STC 0360/RT 01 |
| chloroquine | HICL 04147 |
| hydroxychloroquine | HICL 04151 |
| mycophenolate mofetil | HICL 10012 |
| mycophenolic acid | HICL 25201 |
| azathioprine | HICL 04523 |
| mercaptopurine | HICL 03908 |
| methotrexate | HICL 03905 |
| methotrexate | HICL 24819 |

At 612, control selects the first identified patient from the list of identified candidate patients matching at least one of the medical claims data associated with the target medical condition of the clinical trial, and the prescription claims data associated with treatment of the target health condition the clinical trial.

The system controller 408 is configured to identify whether patients are within a specified age range, at 616. For example, the controller may access demographic information for the patient and determine whether the patient's age is within a target age range for the clinical trial study, such as within a range of age 18 to 70 (or higher or lower boundaries). Other suitable demographic information and boundaries may be used in other example embodiments.

If the patient's age is not within the specified range at 616, control proceeds to remove the patient from the list of eligible candidate patients at 644. If the patient is within the specified age range at 616, control proceeds to 620 to determine whether the patient has any prescription drug claims for excluded drugs. For example, some prescription drugs may be associated with existing diseases of a patient or other health conditions which the clinical trial organizer does not want participating in the clinical trial. Table 2 below lists example Rx claims which may be excluded from the list of candidate patients. The prescription drug claims in Table 2 are provided for example only, and other example embodiments may use other prescription drug claims associated with other target health conditions.

TABLE 2

| Medication | Parameter |
|---|---|
| ocrelizumab | HICL 44178 |
| rituximab | HICL 16848, |
| | HICL 44378 |
| belimumab (Benlysta) | HICL 37462 |
| anifrolumab (Saphnelo) | HICL 47512 |

If there are any excluded prescription drug claims at 620, control proceeds to 644 to remove the patient from the list. If there are no excluded prescription claims for the patient at 620, control proceeds to 624 to determine whether any excluded medical codes are associated with the patient. If there are excluded medical codes present in the patient's medical code history at 624, control proceeds to 644 to remove the patient from the list. If the patient does not have any associated excluded medical codes at 624, control proceeds to 628 to determine whether any excluded health conditions are associated with the patient. Table 3 below lists example medical codes which may be excluded from the list of candidate patients. The medical codes in Table 2 are provided for example only, and other example embodiments may use other medical codes associated with other target health conditions.

TABLE 3

| Medication | J code |
|---|---|
| ocrelizumab | J2350 |
| rituximab | J9310, J9311, J9312 |
| belimumab (Benlysta) | J0490 |
| anifrolumab (Saphnelo) | J0491 |

If the patient has one or more excluded health conditions at 628, control proceeds to remove the patient from the list at 644. If the patient does not have any associated excluded health conditions at 628, control proceeds to 632 to determine whether the patient meets eligibility criteria. For example, control may determine whether the patient is currently covered by health insurance, whether an amount of coverage provided by the health insurance covers the target health condition of the clinical trial, etc. Table 4 below lists example medical claims data which may be excluded from the list of candidate patients. The medical claims data in Table 4 are provided for example only, and other example embodiments may use other medical claims data associated with other target health conditions.

TABLE 4

| Condition | Diagnosis Code |
|---|---|
| Human immunodeficiency virus [HIV] disease | B20 |
| aquired absence of spleen | Z90.81 |
| HIV | B20 |
| Acute Hepatitis B | B16* |
| Acute Hep C | B17.1* |
| Chronic Hep B or C) | B18.0, B18.1, B18.2 |
| malignancy or history of malignancy except cervical | C00*-C96* except C53*, C44.01, C44.02, |
| cancer, basal cell carcinoma or squamous cell | C44.11*, C44.12*, C44.21*, C44.22*, |
| carcinoma | C44.31*, C44.32*, C44.41, C44.42, |
| | C44.51*, C44.52*, C44.61*, C44.62*, |
| | C44.71*, C44.72*, C44.81, C44.82, C44.91, |
| | C44.92 |

If the patient does not meet eligibility criteria at 632, control proceeds to 644 to remove the patient from the list. If the patient meets the eligibility criteria at 632, control proceeds to 636 to obtain patient location data, such as the zip code of the patient or the patient's residence, GPS coordinates associated with the patient, latitude and longitude data stored data for the patient, etc.

At 640, control determines whether the patient is within a specified distance threshold of the clinical trial study sites, such as within 10 miles, within 25 miles, within 50 miles, within 100 miles, etc. If the patient is within a specified distance threshold at 640, control proceeds to 644 remove the patient from the list of eligible candidate patients.

After removing the patient from the list at 1644, or determining that the patient should not be excluded for any of the exclusion criteria, control proceeds to 648 to determine whether any identified patients remain on the list which have not yet been processed or evaluated. If so, control proceeds to select the next identified patient at 652, and returns to 616 to determine whether the patient has an age within the specified age range.

If control determines at 648 that there are no identified patients remaining to be processed or evaluated, control proceeds to transmit the list of target patients at 656, such as transmitting the eligible candidate patient list to a cloud server, another computer, a mobile device of an administrator, etc. In some examples, the system may display the list of target candidate patients on a user interface (e.g., dashboard map), or may automatically schedule sending of communications to a patient or a provider based on the list of target patients having the target health condition within the boundary distance of the clinical trial study site.

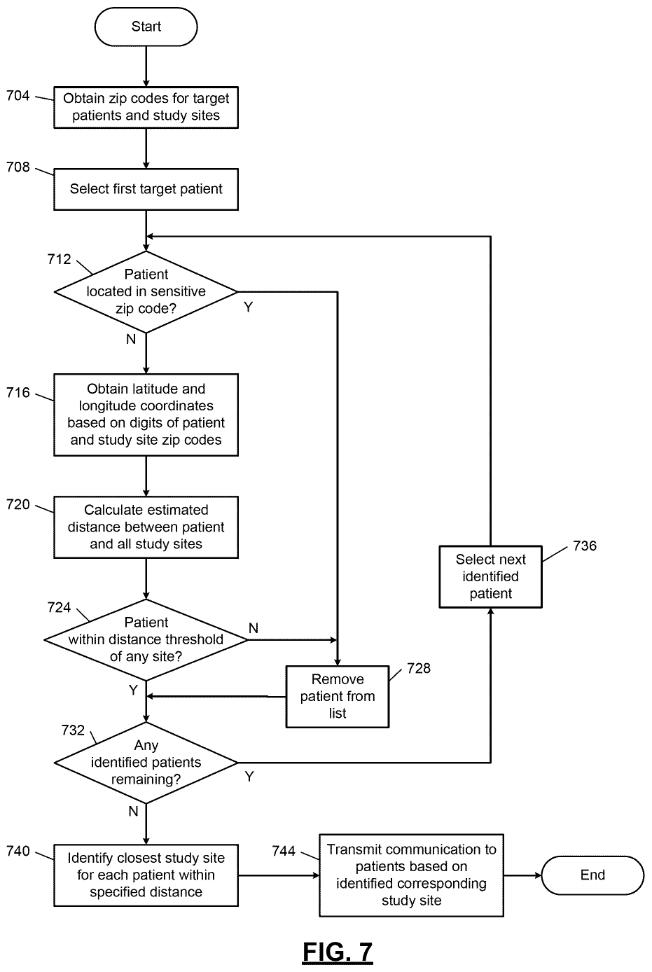

FIG. 7 is a flowchart depicting an example process for determining distances between target patients and clinical trial study sites based on location data. The process illustrated in FIG. 7 may be performed by, for example, the system controller 408 and the patient distance determination module 428.

At line 704, the process begins by obtaining zip codes for target patients and study sites. Control then selects the first target patient at 708. At 712, control determines whether the patient is located in a sensitive zip code. For example, some zip codes may be designated as sensitive areas due to a small size or population (e.g., which may make it possible to identify a patient based on the zip code), for political reasons, demographics, socioeconomic factors, etc. Table 5 below lists example sensitive zip codes with the last two digits masked. The masked zip codes in Table 5 are provided for example only, and other example embodiments may use other sensitive zip code values.

TABLE 5
Zip Code
036
059
063
102
203
556
692
790
821
823
830
831
878
879
884
890
893

If the patient is located in a sensitive zip code at 712, control proceeds to 728 to remove the patient from the list. If the patient is not located in a sensitive zip code at 712, control obtains latitude and longitude coordinates based on, for example, masked digits of zip codes of the patient and one or more study sites. For example, in various implementations the system may mask a last digit of the zip code, the last two digits, or the last three digits.

At 720, control calculates an estimated distance between the patient and all study sites. For example, a cosine similarity function may be used to generate estimated distance between the patient and various study sites. At 724, control determines whether the patient is within a distance threshold of any site. If not, control proceeds to 728 to remove the patient from the list. An example cosine similarity function may be used to estimate a straight line distance between two points on Earth [(latitude1, longitude 1) & (latitude2, longitude2)]. For example, the cosine similarity function may be distance=<radius of earth>*acos((sin(latitude1)*sin(latitude2))+(cos(latitude1)*cos(latitude2)*cos(longitude1−longitude2))).

After removing the patient list 728, or if the patient lives within the distance threshold at 724, control proceeds to 732 to determine whether any identified patients remain for processing or distance evaluation. If so, control proceeds to 738 to select the next identified patient, and then returns to 712 to check whether the next selected patient is within a sensitive zip code.

If control determines at 732 that all identified patients have been processed, control proceeds to 740 to identify the closest study site for each patient within the specified distance threshold. For example, control may use the cosine similarity function to determine which one of multiple study sites is closest to an identified candidate patient.

At 744, control transmits communications to patients, based on an identified corresponding study site. For example, the system may automatically transmit an e-mail, automatically send a letter, automatically transmit a message via a text message or an automated phone call, etc.

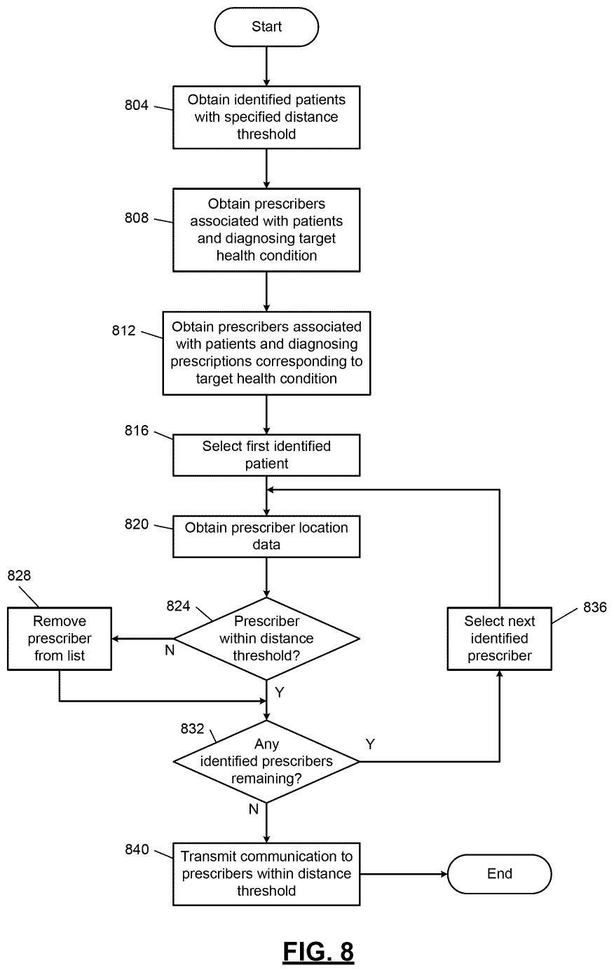

FIG. 8 is a flowchart depicting an example process for identifying target prescribers using a location based dashboard. The process illustrated in FIG. 8 may be performed by, for example, the system controller 408 and the provider evaluation module 430.

The process begins at 804 by the provider evaluation module 430 obtaining identified patients within a specified distance threshold. For example, the identified patients may include patients determined as meeting a target health condition diagnosis, eligibility and location criteria, as determined according to the example process of FIGS. 6 and 7.

At 808, control obtains prescribers associated with the list of patients from 804. The obtained prescribers at 808 may be limited to prescribers which have diagnosed the target health condition (e.g., a target disease for a clinical trial), within a specified period of time (e.g., within the last six months, within the last year, within the last five years, etc.). The selected prescribers may include a minimum number of diagnoses provided by the prescriber, such as diagnosing the target health condition for at least three patients within a specified time period, at least ten patients within a specified time period, etc.

At 812, the system controller is configured to obtain prescribers associated with the list of patients, which have written a prescription for medication corresponding to treatment of the target health condition. The selected prescribers may include a minimum number of prescriptions proscribed by the prescriber, such as prescribing medication associated with treating the target health condition for at least three patients within a specified time period, at least ten patients within a specified time period, etc.

The system controller is configured to select a first identified patient at 816. Control then obtains prescriber location data at 820. The prescriber location data may include, for example, latitude and longitude coordinates for the prescriber, global positioning system (GPS) coordinates for the prescriber, zip code data for the prescriber (which may be masked or unmasked), etc.

At 824, control determines whether the prescriber is within a distance threshold, such as within fifty miles of a clinical trial study site location, within ten miles, within 100 miles, etc. If the prescriber is not located within the specified distance threshold at 824, the prescriber is removed from the prescriber candidate list at 828.

If the prescriber is located within the distance threshold, control proceeds to 832 to determine whether any identified prescribers are remaining in the list that have not yet been evaluated. If so, control selects a next identified prescriber at 836, then returns to 820 to obtain prescriber location data for the next identified prescriber. Once there are no remaining prescribers to process at 832, control proceeds to 840 to transmit a communication to prescribers within the distance threshold.

In various implementations, the system may modify database entries or database entities in response to completing processing of the prescriber list (e.g., by storing or updating records indicating all identified prescribers within a specified distance that were not excluded). The system may update a user interface based on the completed prescriber list, transmit the completed prescriber list via wired or wireless communication to another computing device or mobile computing device, etc. The transmitted communications may include details of the clinical trial and nearest study site for each identified prescriber, an invitation to participate in the clinical trial, or for patients of the prescriber to participate in the clinical trial, etc.

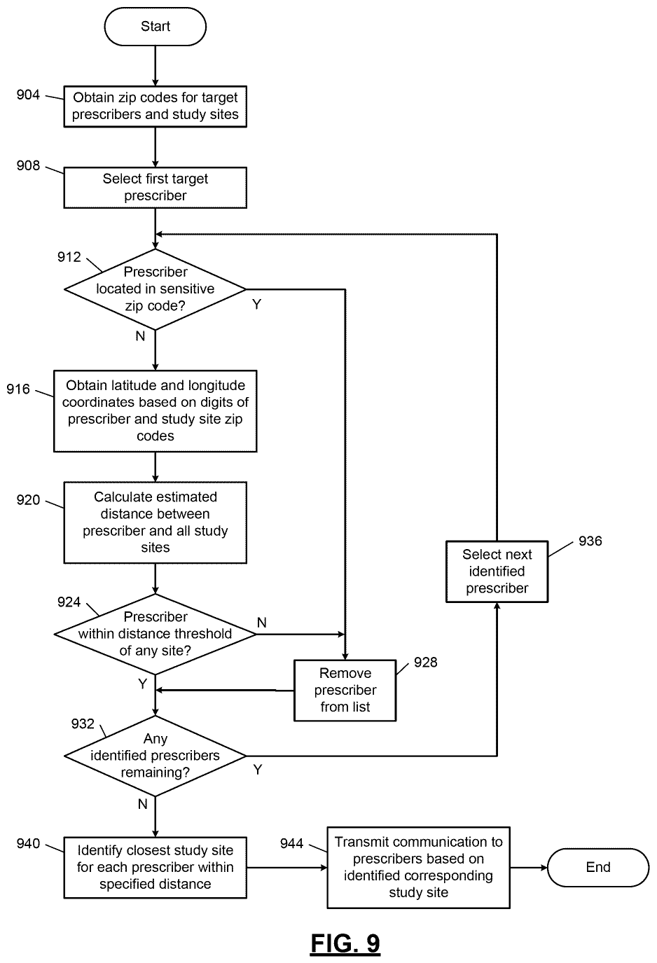

FIG. 9 is a flowchart depicting an example process for determining distances between target prescribers and clinical trial study sites based on location data. The process illustrated in FIG. 9 may be performed by, for example, the system controller 408 and the provider distance determination module 432.

At 904, the process begins by obtaining zip codes for target prescribers and study sites. The zip code data may be unmasked or masked, such as by masking the last two (or more or less) digits of the zip code associated with each prescriber and each study site.

At 908, control selects a first target prescriber. Control then determines at 912 whether the prescriber is located in a sensitive zip code (e.g., a zip code flagged for exclusion from the clinical trial). If the prescriber is located in the sensitive zip code at 912, the prescriber is removed from the list at 928.

If the system controller determines at 912 that the prescriber is not located in a sensitive zip code, control proceeds to 916 to obtain latitude and longitude coordinates based on digits of the prescriber zip code and the study site zip code (e.g., only a first three zip code digits, only a first four zip code digits, etc.).

At 920, the system controller is configured to calculate an estimated distance between the prescriber and all study sites. The distance may be calculated using any suitable algorithm, such as a cosine similarity between the zip code locations of the prescriber and each study site.

The system controller is configured to determine, at 924, whether the prescriber is located within a specified distance threshold (e.g., within ten miles, within fifty miles, etc.) of any study site of the clinical trial. If not, control removes the prescriber from the list at 928.

At 932, control determines whether any identified prescribers from the list are remaining (e.g., have not yet been processed). If so, control selects a next identified prescriber at 936, and returns to 912 to determine whether the next identified prescriber is located in a sensitive zip code.

Once all prescribers have been processed at 932, control proceeds to 940 to identify a closest study site for each prescriber that was located within the specified distance threshold. For example, control may identify which study site has a smallest distance to the prescriber based on the cosine similarity. At 944, control transmits a communication to prescribers based on the identified corresponding study site. The communication may include, for example, information about the clinical trials conducted at the study site, an invitation for the prescriber or patients of the prescriber to participate in the clinical trial at the nearest study site, etc.

### CONCLUSION

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. In the written description and claims, one or more steps within a method may be executed in a different order (or concurrently) without altering the principles of the present disclosure. Similarly, one or more instructions stored in a non-transitory computer-readable medium may be executed in different order (or concurrently) without altering the principles of the present disclosure. Unless indicated otherwise, numbering or other labeling of instructions or method steps is done for convenient reference, not to indicate a fixed order.

Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements.

The phrase “at least one of A, B, and C” should be construed to mean a logical (A OR B OR C), using a non-exclusive logical OR, and should not be construed to mean “at least one of A, at least one of B, and at least one of C.” The term “set” does not necessarily exclude the empty set. The term “non-empty set” may be used to indicate exclusion of the empty set. The term “subset” does not necessarily require a proper subset. In other words, a first subset of a first set may be coextensive with (equal to) the first set.

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are IEEE Standard 802.15.4 (including the ZIGBEE standard from the ZigBee Alliance) and, from the Bluetooth Special Interest Group (SIG), the BLUETOOTH wireless networking standard (including Core Specification versions 3.0, 4.0, 4.1, 4.2, 5.0, and 5.1 from the Bluetooth SIG).

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module. For example, the client module may include a native or web application executing on a client device and in network communication with the server module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. Such apparatuses and methods may be described as computerized apparatuses and computerized methods. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A computer system comprising:
memory hardware configured to store one or more databases, the one or more databases including patient medical claims data and patient prescription claims data; and
processor hardware configured to execute instructions to
obtain a target health condition, wherein the target health condition is a subject of a clinical trial,
access the patient medical claims data and the patient prescription claims data stored in the one or more databases,
identify a set of candidate patients having at least one medical claim associated with the target health condition, based on the patient medical claim data,
update the set of candidate patients to include patients having at least one prescription claim associated with treatment of the target health condition, based on the patient prescription claim data,
for each candidate patient in the set of candidate patients,
remove the candidate patient from the set in response to the candidate patient having demographic data which matches demographic exclusion criteria,
remove the candidate patient from the set in response to the candidate patient having medical history data which matches medical exclusion criteria,
remove the candidate patient from the set in response to health insurance coverage data of the candidate patient failing to satisfy health insurance eligibility criteria,
determine a distance between a location of the candidate patient and a location of at least one study site of the clinical trial, and
remove the candidate patient from the set when the distance is greater than a specified distance threshold,
store a set of eligible candidate patients after selective removal of candidate patients based on the demographic exclusion criteria, the medical exclusion criteria, the health insurance eligibility criteria and the specified distance threshold,
transmit a communication to at least one computing device, the communication including the set of eligible candidate patients, and
display the set of eligible candidate patients and the at least one study site on a dashboard map of a user interface.

2. The computer system of claim 1, wherein the processor hardware is configured to execute instructions to remove the candidate patient from the set in response to the candidate patient having medical history data which matches medical exclusion criteria by:
comparing prescription claims data of the candidate patient to a list of excluded prescription claims;
comparing medical codes associated with the candidate patient to a list of excluded medical codes; and
comparing medical claims data of the candidate patient to a list of excluded medical claims.

3. The computer system of claim 2, wherein the list of excluded prescription claims includes a prescription drug code for at least one of ocrelizumab, rituximab, belimumab, or anifrolumab.

4. The computer system of claim 2, wherein the list of excluded medical claims includes a medical claim code for at least one of human immunodeficiency virus disease, acquired absence of spleen, acute hepatitis B, acute hepatitis C, chronic hepatitis B, chronic hepatitis C, or history of malignancy except cervical cancer, basal cell carcinoma, squamous cell carcinoma or anifrolumab.

5. The computer system of claim 1, wherein identifying a set of candidate patients having at least one prescription claim associated with treatment of the target health condition includes a prescription drug code for at least one of oral corticosteroids, chloroquine, hydroxychloroquine, mycophenolate mofetil, mycophenolic acid, azathioprine, mercaptopurine, or methotrexate.

6. The computer system of claim 5, wherein the target health condition is Systemic Lupus Erythematosus.

7. The computer system of claim 1, wherein determining the distance between the location of the candidate patient and the location of at least one study site includes using a cosine similarity function.

8. The computer system of claim 1, wherein the specified distance threshold is less than or equal to fifty miles.

9. The computer system of claim 1, wherein demographic exclusion criteria includes a patient age range of greater than or equal to eighteen years of age and less than or equal to seventy years of age.

10. The computer system of claim 1, wherein:
the location of the candidate patient and the location of at least one study site are masked zip code locations; and
the masked zip code locations have the last two digits of each zip code masked.

11. The computer system of claim 1, wherein the processor hardware is configured to, for each candidate patient in the set of candidate patients, remove the candidate patient from the set in response to the candidate patient having a zip code matching a list of specified sensitive zip codes.

12. The computer system of claim 1, wherein the processor hardware is configured to execute instructions to:
obtain a set of providers associated with the set of eligible candidate patients;
identify a set of candidate providers having at least one medical claim diagnosis associated with the target health condition, based on provider diagnosis data; and
update the set of candidate providers to include providers having proscribed at least one prescription claim associated with treatment of the target health condition, based on provider prescription data.

13. The computer system of claim 12, wherein the processor hardware is configured to execute instructions to, for each candidate provider in the set of candidate providers:
determine a distance between a location of the candidate provider and the location of the at least one study site of the clinical trial; and
remove the candidate provider from the set when the distance is greater than the specified distance threshold.

14. A method for implementing location based dashboards for clinical trials, the method comprising:
obtaining a target health condition, wherein the target health condition is a subject of a clinical trial;
accessing patient medical claims data and patient prescription claims data stored in one or more databases;
identifying a set of candidate patients having at least one medical claim associated with the target health condition, based on the patient medical claim data;
updating the set of candidate patients to include patients having at least one prescription claim associated with treatment of the target health condition, based on the patient prescription claim data;
for each candidate patient in the set of candidate patients,
removing the candidate patient from the set in response to the candidate patient having demographic data which matches demographic exclusion criteria,
removing the candidate patient from the set in response to the candidate patient having medical history data which matches medical exclusion criteria,
removing the candidate patient from the set in response to health insurance coverage data of the candidate patient failing to satisfy health insurance eligibility criteria;
determining a distance between a location of the candidate patient and a location of at least one study site of the clinical trial, and
removing the candidate patient from the set when the distance is greater than a specified distance threshold;
storing a set of eligible candidate patients after selective removal of candidate patients based on the demographic exclusion criteria, the medical exclusion criteria, the health insurance eligibility criteria and the specified distance threshold;
transmitting a communication to at least one computing device, the communication including the set of eligible candidate patients; and
displaying the set of eligible candidate patients and the at least one study site on a dashboard map of a user interface.

15. The method of claim 14, wherein removing the candidate patient from the set in response to the candidate patient having medical history data which matches medical exclusion criteria includes:
comparing prescription claims data of the candidate patient to a list of excluded prescription claims;
comparing medical codes associated with the candidate patient to a list of excluded medical codes; and
comparing medical claims data of the candidate patient to a list of excluded medical claims.

16. The method of claim 15, wherein the list of excluded prescription claims includes a prescription drug code for at least one of ocrelizumab, rituximab, belimumab, or anifrolumab.

17. The method of claim 15, wherein the list of excluded medical claims includes a medical claim code for at least one of human immunodeficiency virus disease, acquired absence of spleen, acute hepatitis B, acute hepatitis C, chronic hepatitis B, chronic hepatitis C, or history of malignancy except cervical cancer, basal cell carcinoma, squamous cell carcinoma or anifrolumab.

18. The method of claim 14, wherein identifying a set of candidate patients having at least one prescription claim associated with treatment of the target health condition includes a prescription drug code for at least one of oral corticosteroids, chloroquine, hydroxychloroquine, mycophenolate mofetil, mycophenolic acid, azathioprine, mercaptopurine, or methotrexate.

19. The method of claim 18, wherein the target health condition is Systemic Lupus Erythematosus.

20. The method of claim 14, wherein determining the distance between the location of the candidate patient and the location of at least one study site includes using a cosine similarity function.
